(19) United States
(12) Patent Application Publication
(10) Pub. No.: US 2021/0209549 A1
(43) Pub. Date: Jul. 8, 2021

(54) SYSTEMS AND METHODS FOR AUTOMATED TRACKING AND OPTIMIZATION OF GLOBAL MANUFACTURING AND SUPPLY BASED ON IMPACTS OF POST-APPROVAL CHANGES

(71) Applicant: Millennium Pharmaceuticals, Inc., Cambridge, MA (US)

(72) Inventors: Clare Elizabeth Bornstein, Stonington, CT (US); Kevin McDonald, Littleton, MA (US); Lam Raga Anggara Markely, Cambridge, MA (US)

(21) Appl. No.: 17/059,026

(22) PCT Filed: May 28, 2019
(86) PCT No.: PCT/US2019/034181

§ 371 Date: Nov. 25, 2020

Related U.S. Application Data
(60) Provisional application No. 62/677,163, filed on May 28, 2018.

(51) Int. Cl. G06Q 10/08; G06F 16/22; G06N 20/00; G16H 70/40

(52) U.S. Cl. CPC G06Q 10/0833; G16H 70/40; G06N 20/00; G06F 16/22

(57) ABSTRACT

A computing device-implemented method for automated tracking and optimization of global manufacturing and supply based on impacts of post-approval changes (PACs) is discussed. An exemplary method includes retrieving data from data sources including at least an inventory database, a regulatory database, and a PAC information database. The method further includes analyzing the retrieved data to identify one or more impacts of at least one PAC on one or more drug products or drug substances using a predefined ruleset for determining which changes were implemented for each lot. The method also includes generating a list of the identified one or more impacts on the one or more drug products or drug substances.

### CROSS-REFERENCE TO RELATED APPLICATIONS

This application claims priority to and the benefit of U.S. Provisional Patent Application No. 62/677,163, filed May 28, 2018, the disclosure of which is incorporated herein by reference in its entirety.

### BACKGROUND

Pharmaceutical and biotechnology companies routinely make post-approval changes (PACs) and updates to a drug's manufacturing processes following an initial regulatory approval of the drug. The changes may be changes initiated by the manufacturer after an initial regulatory approval or may be commitments required as part of the initial regulatory approval. Some PACs require a subsequent post-change approval by a national regulatory authority of each country before a company can deliver a product manufactured using the changed processes.

### BRIEF DESCRIPTION OF DRAWINGS

To assist those of skill in the art in making and using a system for automated tracking and optimization of PACs and associated methods, reference is made to the accompanying figures. The accompanying figures, which are incorporated in and constitute a part of this specification, illustrate one or more embodiments and, together with the description, help to explain the embodiments. Illustrative embodiments are shown by way of example in the accompanying drawings and should not be considered as limiting. In the figures:

FIG. 1 illustrates an exemplary network environment suitable for a system for automated tracking and optimization of post-approval changes, in accordance with an exemplary embodiment;

FIG. 2 illustrates an exemplary method for processing data obtained from databases and displaying results on a dashboard using an ATOMS module, according to an exemplary embodiment;

FIG. 3 illustrates a method to determine filing estimations, according to an exemplary embodiment;

FIG. 4 illustrates a method to generate a shipping list, according to an exemplary embodiment;

FIG. 5 is a block diagram of an example computing device that can be used to perform one or more steps of the methods provided by exemplary embodiments;

FIG. 6 is a method for automated tracking and optimization of global manufacturing and supply based on PAC impact, in accordance with an exemplary embodiment;

FIG. 7 illustrates a first sample graphical user interface displaying lots that are invalid on a future date as a result of changes implemented during manufacturing, in accordance with an exemplary embodiment;

FIG. 8 illustrates a second sample graphical user interface displaying lots that are invalid on a future date as a result of changes implemented during manufacturing, in accordance with an exemplary embodiment;

FIG. 9 illustrates a sample graphical user interface displaying a length of time until lots are consumed, in accordance with an exemplary embodiment;

FIG. 10 illustrates a sample graphical user interface displaying lots impacted by post-approval changes, in accordance with an exemplary embodiment;

FIG. 11 illustrates a sample graphical user interface displaying pending post-approval changes in select countries, according to an exemplary embodiment;

FIG. 12 illustrates a sample graphical user interface displaying statuses of post-approval changes in select countries, according to an exemplary embodiment;

FIG. 13 illustrates a sample graphical user interface displaying post-approval changes that are missing a date of change implementation in select countries, according to an exemplary embodiment;

FIG. 14 illustrates a sample graphical user interface displaying an execution of the analysis process performed by an ATOMS module, according to an exemplary embodiment;

FIG. 15 is a method for automated tracking and optimization of global manufacturing and supply based on PAC impact, in accordance with an exemplary embodiment; and

FIG. 16 is a method for automated tracking and optimization of global manufacturing and supply based on impacts of PACs, in accordance with an exemplary embodiment.

### DETAILED DESCRIPTION

The problem of drug shortages is recognized globally and one contributing factor to shortages is the challenge of managing post-approval changes (PACs). PACs are necessitated by product lifecycle management activities, such as adding a new manufacturing site to meet increased product demand, or modifying a manufacturing process to improve product yield. These challenges are driven by the complexity of a global regulatory environment where the PAC approval process can be anywhere from less than six months to greater than 18 months. Ideally, PACs enable a sponsor to bring later developed manufacturing strategies forward that deliver patient-centric value. However, it is difficult to support an environment of continuous improvement given the regulatory uncertainty associated with varying global submission timelines.

Additionally, once a PAC has been authorized for a product, lots produced with the PAC are required by the regulatory authority of a given market to be evaluated by a quality assurance group within a pharmaceutical organization to be sure the implemented changes used to produce the lot were approved for the market to which the lot is delivered.

An additional factor contributing to drug shortages is the tracking of lots affected by PACs. A lot is a particular quantity of a drug product or drug substance, typically from a single manufacturer and is identified by a lot number. The lot number is an identifier assigned to the particular lot of the drug product or drug substance. Lots affected by PACs may be released into a destined market following regulatory approval updates about the lots entered into a Quality Management System (QMS), such as Trackwise Quality Management Software™ or a combination of CATSWeb Quality Management Software™ and Liquent InSight Regulatory Information Management Software™, holding information regarding the approval status of each PAC within the different markets. As the manufacturing volume and PAC volume increase, there is a geometric growth in the total number of PAC evaluations necessary to release the affected lots.

Supply planners face a parallel challenge because they must determine if a given lot allocated to a country is prohibited by the PAC regulatory status in the market. A PAC regulatory status prohibiting the release of a lot to a country may include the status categories: “submission incomplete,” “submission under review,” “submission rejected,” or “change cannot be implemented,” or similar terms. If the PAC has not been approved by the regulatory authority of the market, the product cannot be sold. An information gap exists between databases holding regulatory updates such as, but not limited to, Liquent InSight Regulatory Information Management Software™, databases holding PAC information such as, but not limited to, CATSWeb Quality Management Software™ or Trackwise Quality Management Software™, and databases holding product inventory data, such as, but not limited to, SAP Enterprise Resource Planner™ or Oracle Enterprise Resource Planner™, and is a consequence of the various systems not being linked. Similar problems exist when relying on other non-linked databases such as databases holding PAC-related information. As a result, the total number of PAC evaluations for each drug product or drug substance lot grows geometrically with the number of approved markets. Ensuring the product remains compliant through the various country approvals becomes a substantial risk when there is insufficient transparency between the PAC, regulatory, and supply chain systems. As a result, the compliance pressure on the product increases as the product transitions from having a single manufacturing process, supply chain, with a common technical document (CTD), and indication to one with multiple manufacturing processes, a diversified supply chain, multiple CTDs, and potentially multiple indications through its lifecycle. A CTD details the specifications of a given product to be sold, including prescribing information, pharmaceutical documentation, pharmacology and toxicology data and safety and efficacy data. A CTD is prepared on a per-market basis when a product is registered for sale in a given country. Over a product's lifetime, the product may be registered for additional indications, or as a method of therapy for additional illnesses or conditions. Over time, the number of CTDs and indications increases significantly due to an increased number of markets where the product is sold.

Systems and methods are provided herein for automated tracking and optimization of global manufacturing and supply based on impacts of PACs, in accordance with exemplary embodiments. In one embodiment, a system includes a computing device equipped with a processor that is communicatively coupled to a storage device. The computing device is configured to execute an Automated Tracking and Optimization for global Manufacturing and Supply (ATOMS) module. In some embodiments, the ATOMS module may further include an artificial intelligence (AI) and/or machine-learning module (hereafter AI/ML). In one embodiment, the computing device executing the ATOMS module is communicatively coupled to three or more databases or data repositories. In an exemplary embodiment, the computing device is coupled to a database storing product inventory data, a database storing PAC-related data including data for a change control process implementing a PAC into a production process, and a database storing regulatory data. In one embodiment, the stored data is distributed among different formal software systems, static file inputs, or informal ad-hoc data repositories.

Data sources connected to the ATOMS module are centrally maintained by the organization that owns the product to be analyzed. Inventory, regulatory, and PAC databases are all managed, maintained, and updated internally. Inventory data is maintained and updated by the supply chain and quality groups within an organization. While external inventory data may be individually maintained by external manufacturing sites in other sources, the data is replicated into internal databases. PAC data is maintained and updated by the supply chain, quality and technical operations groups within an organization. Regulatory data is maintained and updated by the regulatory affairs group within an organization which retrieves the data from the relevant regulatory authorities of a country.

In embodiments, the ATOMS module improves the process of tracking PACs as they relate to existing and planned manufacturing of drug substance and/or drug product lots by extracting key information from PAC and regulatory databases, and an inventory database. Using a predefined rule set, the ATOMS module determines what PACs were implemented during the manufacture of each lot. The ATOMS module extracts lot information, such as lot number, date of manufacture, and manufacturing facility, from the inventory database or data source. The ATOMS module extracts PAC information for a manufacturing process or material supply, such as information related to a Contract Manufacturing Organization (CMO) or internal manufacturing site affected, material affected, equipment affected, and date of implementation, from the PAC database or data source. A CMO or external manufacturer may be contracted for use in a manufacturing supply chain when internal manufacturing capabilities are not available. The ATOMS module also extracts regulatory information, such as regulatory filing status of a PAC, regulatory dispatch date, regulatory submission date, and regulatory approval date, from the regulatory database or data source. The ATOMS module compares the information retrieved from the three data sets and generates a list of applicable post-approval PACs, as described herein.

In some embodiments, the ATOMS module tracks and evaluates applicable PACs with respect to the global supply chain on a country-by-country basis. For example, the ATOMS module may evaluate the regulatory filing status of each PAC implemented during the manufacture of a given lot, in each global market. The ATOMS module determines and tracks whether the appropriate national regulatory authority has approved each of the PACs. The ATOMS module further ascertains which countries can accept a drug substance and/or drug product based on the regulatory evaluation of PACs implemented during manufacture.

In some embodiments, the ATOMS module evaluates finished drug product lots for PAC impact at each step in the supply chain. Each component of the finished drug product lot, such as active pharmaceutical ingredient (API) or bulk drug substance (BDS), is evaluated for PAC impact and subsequent regulatory compliance in the manners described herein. The ATOMS module aggregates the PACs implemented on each component combined in a downstream manufacturing process. The ATOMS computing module reports applicable PACs for both individual component batches and downstream batches combining one or more components.

In one embodiment, the ATOMS module uses a predefined ruleset to evaluate PAC impact on various materials manufactured at varying stages in a product's supply chain. These rules include, but are not limited to, assessing the manufacturing site at which the lot was produced, the equipment utilized during manufacture and the processes utilized during manufacture, and comparing this information with those indicated as modified or proposed by the PAC. PACs may impact one or more CMOs, internal manufacturing sites, equipment, or processes. PACs are implemented into manufacturing processes at each manufacturing site and the dates of implementation are recorded. The ATOMS module collects genealogy information pertaining to the material being analyzed, such as bulk drug substance or drug product. This genealogy establishes the date of manufacture, site of manufacture, and applicable equipment or processes of each material making up the lot being evaluated for impact by a PAC. The ATOMS module determines impact by matching affected manufacturing sites, equipment, and processes as indicated by a given PAC, and the genealogy information of the lot being analyzed. If the PAC was implemented at a site indicated prior to the date of manufacture of the material, the ATOMS module ascertains that the PAC impacted the manufacture of the lot being analyzed. This predefined ruleset may be leveraged in the embodiments described throughout this document to determine applicable PACs.

In one embodiment, the ATOMS module uses set notation to compare the changes impacting each lot with those approved in each country. The set notation is a comparative mechanism that ascertains whether a given lot can be shipped to a given country. For example, a set notation may be: {‘Active’: [31377, 31380, 32797]}—{‘Approved’: [31377, 31380, 31642, 31682, 32797, 33342]}={ }, indicating no PACs prohibit release of the product for the selected market. For a converse example, a set notation may be: {‘Active’: [31377, 31380, 32797, 52398]}−{‘Approved’: [31377, 31380, 31642, 31682, 32797, 33342]}={52398}, indicating PAC number 52398 is prohibiting release of product for the selected market. The results determine which lots of a drug are fully compliant from a regulatory standpoint. If a change is implemented but not approved in a given country, the ATOMS module identifies the unapproved PACs along with market specific compliant drug product quantities to inform a new regulatory submission strategy and reduce the risk of drug shortages. In an embodiment, the set notation result is translated into a barcode for a lot. In an embodiment, the set notation result populates the list of lots that can be sent to a country.

In embodiments, the ATOMS module may perform analysis for each country in which the product is approved and, using the aggregate results, determine whether the product is compliant across the global market. The ATOMS module may also determine outstanding (i.e.: not yet approved) PACs across the global market, thereby informing new regulatory submission strategies. In an exemplary embodiment, the ATOMS module completes the analysis of the information in less than 30 seconds to ensure that stakeholders across the functional business units of an entity providing tracked products or substances have timely compliance status information.

In addition to tracking and determining compliance, in one embodiment, the ATOMS module estimates when to file PACs in countries that prohibit pre-change material once new changes are approved. By estimating a stock rundown time, as well as an average approval timeframe, the ATOMS module determines an optimum filing window for new PACs that allow for complete depletion of old stock prior to the introduction of new stock.

In an embodiment, the ATOMS module uses intelligent allocation to optimize where valid drug products should be shipped based on monthly consumption rates and available stock by indication of a confidence value. For a given country, the ATOMS module may determine simple consumption time (t) by dividing drug product (DP) Units (u) by consumption rate (c). The intelligent allocation process serves to equalize simple consumption time across valid countries with respect to a forecast risk factor. For a given country, the ATOMS module may extend simple consumption time to determine time to consume product with respect to a forecast risk factor, where consumption time (t)=DP Units (u)/(consumption rate (c)*consumption error (E_{c})). The ATOMS module allocates available drug product units to each PAC compliant country such that consumption time is equalized, where for all PAC-compliant countries (p): Eq(t):={u/(c*E_{c}), c∈p}. Thus, the ATOMS module may allocate drug product units to each PAC compliant country such that consumption time is equalized with respect to the forecast risk factor.

The ATOMS module may perform a forecast risk factor assessment that includes consumption rate and approval time. The forecast risk factor of consumption rate may be a historical average of discrepancy in recorded data set, where consumption error (E_{c})={x̅|f(x)=Forecasted Rate of Consumption (c₀)/Actual Rate of Consumption (c₁)}. The forecast risk factor of approval time may be a historical average of discrepancy in recorded data set, where approval error (Eₐ)={x̅|f(x)=Forecasted Time for Approval (a₀)/Actual Time for Approval (a₁)}. The forecast risk factor may be used to determine filing dates as well as adjustments to inventory in the respective markets having a high-risk factor.

In an embodiment, the forecast risk factor of consumption rate may be used when calculating the time to consume an allocation of drug product in a market—this is primarily because the consumption forecasts for each country are of varying accuracy. If overestimation or underestimations are chronically made in a country market, the consumption time may be calculated with respect to that trend. Similarly, when calculating an optimum filing date, the consumption forecast risk and the approval time forecast risk are factored in. If overestimation or underestimations are chronically made regarding how long it takes a country to approve a PAC, the filing window may be calculated with respect to that trend, in addition to the consumption time forecast risk.

In an embodiment, the ATOMS module may further use intelligent filing to determine an earliest date and a latest date to file for approval of changes given an allocation of drug product in a given country market. Intelligent filing establishes a by-country target filing date and by-country filing window with respect to the forecast risk factor of consumption and the forecast risk factor of approval. The by-country target filing date determines a best-case scenario filing date assuming a perfect consumption forecast, where the target filing date may be expressed as (d₀)=t−(a₀*Eₐ). The by country filing window finds the earliest date and the latest date to file, establishing an optimum window, expressed as the minimum filing date (d₁)=t−(a₀*Eₐ)−(E_{c}*grace period (g)), and the maximum filing date (d₂)=t−(a₀*Eₐ)+(E_{c}*g).

In one embodiment, the ATOMS module (uses an AI/ML module) to analyze stock component lots that have yet to be processed into downstream patient deliverables. The ATOMS module uses the AI/ML module to preemptively ascertain the impact of PACs on upstream component lots and determine which upstream component lots should have their shipment downstream be accelerated for manufacturing so as to address demand in countries nearing stock out. Additionally, by identifying the PAC filing status of each ingredient in a finished drug product, prior to manufacturing of the ingredient into drug product, the ATOMS module can use artificial intelligence and machine learning to select optimal component lots to minimize the amount of regulatory action required to release a new batch, especially in countries nearing or projecting a stock out. In one embodiment, the ATOMS module (and in particular the AI/ML module) intelligently plans and schedules future lots to ensure PAC compliance using predictive modeling and optimization.

For example, in an embodiment, the projections from the AI/ML module are utilized to control when to order raw materials for manufacturing lots, taking into account the time of year and time it takes to receive materials as some raw materials are seasonal and/or have convoluted shipping requirements. In another embodiment, the projections from the AI/ML module are utilized to control shipping schedules. In a further embodiment, the projections from the AI/ML module are utilized to control the timing of regulatory filings. In another embodiment projections from the AI/ML module are utilized by the ATOMS module to control country-specific packaging and labeling. In one embodiment, the projections from the AI/ML module may be utilized by the ATOMS module to execute a command to commence manufacturing of a drug substance and/or drug product.

The ATOMS module further generates a regulatory submission strategy for component lots affected by PACs prohibiting release of product. This may be accomplished by determining when the upstream component will be forward processed into downstream product to be shipped to patients. The result allows for improved regulatory submission strategies, earlier interception of potential shortages, and greater line of sight in the consequences of manufacturing lifecycle management decisions.

The ATOMS AI/ML module model is trained on the historical rate of product consumption, historical product distribution, and historical approval timelines for PACs of a given type, as well as rate of forward processing of materials in manufacturing. “Forward processing refers to the utilization of a component lot as an ingredient in manufacturing a downstream material, such as drug substance or drug product. The model yields a historically founded dataset that accurately describes both demand, and ability to meet demand, with respect to PAC approval. To determine which materials should have their shipment downstream be accelerated for manufacturing as to address product demand, the AI/ML module ascertains the likelihood of each available upstream component's PACs being approved in each global market by the time the component is present in product to be shipped to market, considering both demand and manufacturing schedules, and selects the component with greatest chance of approval in each market. This informs manufacturing scheduling to ensure that PACs are approved prior to distribution. This model output can also be used to identify the component lots that will need the least number of PACs to be filed prior to distribution. Additionally, this model output can be utilized in a similar manner so as to identify potential “stock outs” in a given market (“stock outs” refers to a product stock being exhausted). If the model cannot identify an upstream component that has sufficient probability of having all PACs approved prior to distribution, an alert is generated. By using the same model output, the ATOMS AI/ML module generates a regulatory submission strategy for component lots with numerous PACs prohibiting release of product. By using the model to establish when components will appear in product to be shipped to market, as well as the historical timeframes for approval, the AI/ML module suggests an ideal filing timeframe for the PAC in order to ensure that the PAC is approved prior to distribution.

Contract manufacturing organizations (CMOs) and internal manufacturing sites may be directly affected by post-approval changes (PACs). In some embodiments, the ATOMS module establishes the impact of PACs as they pertain to CMOs or internal manufacturing sites. The ATOMS module collects information from a Quality Management System and outputs it in a product-specific format to generate a barcode header. For example, a 1 indicates that the PAC impacts the manufacturing site (identified by an ID number), and a 0 indicates no impact to the manufacturing site, as illustrated in Table 1. The manufacturing sites, as shown in table 1, include manufacturing sites for drug substance (DS-CMOs A, B, C and D), and manufacturing sites for drug product (DP-CMOs A, B, C, D, E, and F).

TABLE 1
Illustrative Example of Impact Barcodes

| | DS-CMO | | | | DP-CMO | | | | | |
|---|---|---|---|---|---|---|---|---|---|---|
| ID | A | B | C | D | A | B | C | D | E | F |
| 34885 | 1 | 0 | 0 | 0 | 0 | 0 | 0 | 0 | 0 | 0 |
| 37183 | 0 | 1 | 0 | 0 | 0 | 0 | 0 | 0 | 0 | 0 |
| 37951 | 0 | 0 | 0 | 0 | 0 | 0 | 0 | 0 | 0 | 0 |
| 39074 | 0 | 0 | 0 | 0 | 0 | 0 | 0 | 0 | 1 | 0 |
| 39217 | 0 | 0 | 0 | 0 | 1 | 0 | 0 | 0 | 0 | 0 |
| 39713 | 0 | 0 | 0 | 0 | 0 | 1 | 1 | 1 | 0 | 0 |

In other embodiments, the set notation result or the list of lots derived from the ruleset can be assigned a reference number which can be translated into a barcode.

The ATOMS module generates the barcode header to describe which CMO or manufacturing site is affected by a given change. A physical barcode may accompany a component through its manufacture at various CMOs or manufacturing sites. Certain CMOs or manufacturing sites also implement different suites/rooms that require separate regulatory action. Each drug component is manufactured at a different facility; each component can be manufactured at one of many different facilities or suites (e.g., Ingredient A can be manufactured at one of 2 facilities, while Ingredient B can be manufactured at one of 4 facilities). Changes often only impact one or some of the facilities, and may impact one or more components. The physical barcode would be ascertained in the predefined ruleset described above.

In another embodiment, the ATOMS module generates batch manufacturing records (e.g., process protocols) based on PACs to be used in manufacturing by CMOs. A batch manufacturing record includes data of a batch manufacturing process. The ATOMS module amends the batch manufacturing records to include the PACs for a particular country. One or more CMOs follow the batch manufacturing records to produce drug ingredients needed to replenish inventory levels in the particular country. The completed lot incorporates the relevant PACs in that country.

In some embodiments, the ATOMS module is communicatively coupled to a first manufacturing device. In one embodiment, the first manufacturing device is and/or includes a product labeler. In such an embodiment, based on the PAC impact determined by the ATOMS module, the ATOMS module may transmit instructions for the first manufacturing device to generate and add an impact label, such as a barcode or barcode header, to one or more lots. As component lots are combined into downstream drug product lots across other CMOs, the impact labels are aggregated as the product moves through the supply chain such that finished product to be shipped to patients is released to the purchasing organization with a record of what PACs were implemented during its manufacture. This label can be used by internal and external regulatory groups to determine PAC filing compliance in world markets.

In another embodiment, the ATOMS module is communicatively coupled to a second manufacturing device. In one embodiment, the second manufacturing device is and/or includes a product labeler. In such an embodiment, the ATOMS module may instruct the second manufacturing device to generate and add a genealogy label, such as a barcode or barcode header, to one or more lots. The genealogy may include, for example, a site of manufacture, the processing suite in use at the site of manufacture, the date of manufacture, and the quantity of components used. As component lots are combined into downstream drug product lots across other CMOs, the genealogy labels are aggregated as the product moves through the supply chain such that finished product to be shipped to patients is released to the purchasing organization with a record of its complete genealogy and manufacturing date stamps.

In a further embodiment, the ATOMS module is also communicatively coupled to a third manufacturing device. In one embodiment, the third manufacturing device is and/or includes an inventory management system. In such an embodiment based on the PAC impact determined by the ATOMS module, the ATOMS module may instruct the third manufacturing device to forward deploy a particular lot in inventory to a particular market or country as to address the market or country nearing stock out due to consumption.

In another embodiment, the ATOMS module is also communicatively coupled to an inventory management device. In one embodiment, the inventory device is and/or includes a stock quantity aggregate system such as an SAP Enterprise Resource Planner. In such an embodiment based on the PAC impact determined by the ATOMS module, the ATOMS module may instruct the inventory management device to report valid stock levels by component and drug product in each market and to update available stock quantities based on allocation as determined by the ATOMS module. In some embodiments, the ATOMS module may update the available stock quantities by automatically ordering components, excipients, and/or drug products from the inventory management device in time to be qualified or incorporated into the manufacturing stream. For example, the ATOMS module may order a particular drug product based on consumption and stockout risk.

In another embodiment, the ATOMS module retrieves expiration dates of components, including expiration dates for drug substance and/or drug product, from a database to ensure that the supply in countries takes into account usage rates and product expirations. The ATOMS module can determine whether an expiration date of a component exceeds a predefined threshold by comparing the expiration date to the predefined threshold (e.g., there is less than 60 days until expiration). The ATOMS module can transmit an alert to prompt manufacturing of a component nearing expiration or prompt shipping supply of the component to a country whose stock is nearing expiration.

The described methods and systems are portable to different drug types and adaptable to different supply chains. The methods and systems are useful for, but not limited to, (single component) small molecule and biologic products as well as multicomponent products such as antibody drug conjugates.

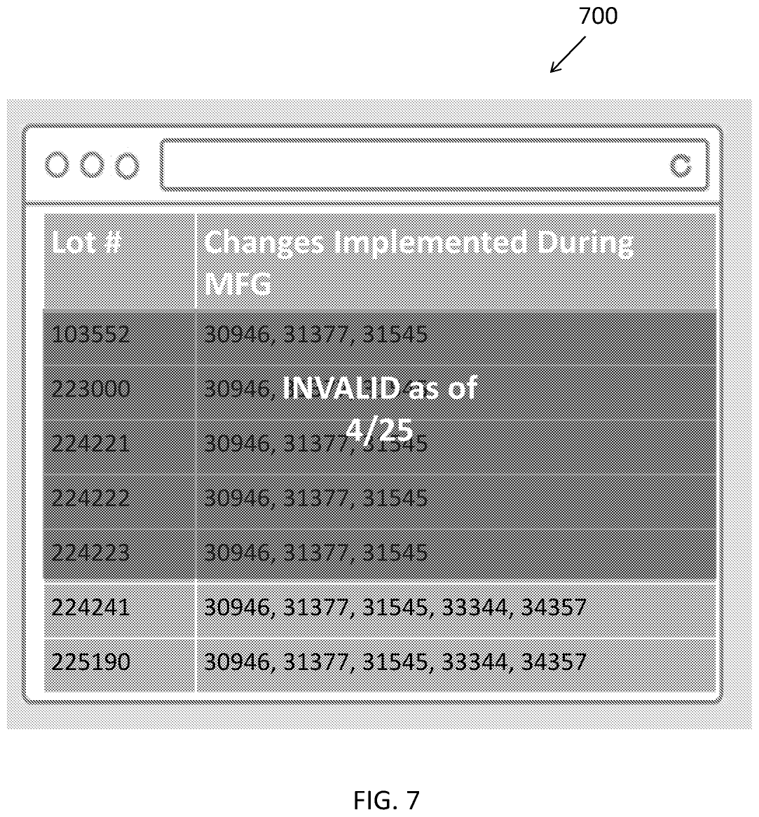

As shown in FIGS. 7-13, in some embodiments the above information may be displayed in a dashboard on a user-computing device. This information may include, but is not limited to, lots that are invalid on a future date as a result of changes implemented during manufacturing, a length of time until lots are consumed, lots impacted by PACs, pending PACs in select countries, PAC statuses in select countries, and/or PACs missing dates of implementation and their filing status in select countries.

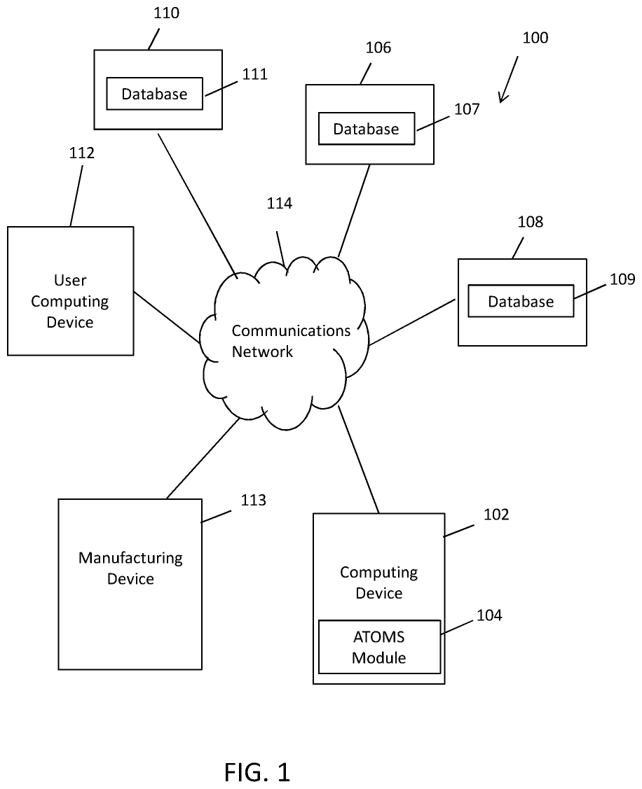

FIG. 1 illustrates an exemplary network environment suitable for a system 100 for automated tracking and optimization of post approval changes, in accordance with an exemplary embodiment. System 100 includes at least one computing device 102 executing an ATOMS module 104, at least one storage device 106 including a first database 107, at least one storage device 108 including a second database 109, at least one storage device 110 including a third database 111, at least one user computing device 112, and at least one manufacturing device 113. The ATOMS module 104 includes one or more applications, processes or other forms of executable code with the functionality described herein for automated tracking and optimization of post approval changes.

In one embodiment, the first database 107 is an inventory database holding information for drug products and/or drug substances that includes drug genealogy, including dates of manufacture of components. An example type of the first database 107 may include an SAP Enterprise Resource Planner. The first database 107 may further include one or more of finished drug product genealogy (all components that make up a finished drug product, such as active product ingredient (API), bulk drug substance (BDS), etc.), the site of manufacture for each component, and the date of manufacture of each component. The ATOMS module 104 extracts data from database 107 in the form of drug genealogy and supply quantities. The ATOMS module 104 also extracts requisite information from database 107 in an automated format and relays the requisite information for processing.

In one embodiment, the second database 109 is a PAC database that includes PAC impact, PAC date of implementation, and PAC approvals. An example type of the second database 109 may include Trackwise Quality Management System. The second database 109 may further contain data regarding one or more of component types or material affected by a PAC, manufacturing sites affected by a PAC, manufacturing suites affected by a PAC, manufacturing equipment affected by a PAC, manufacturing specifications affected by a PAC, date(s) of implementation of a PAC, and regulatory approval of a PAC in world markets. The ATOMS module 104 extracts requisite information from database 109 in an automated format and relays the requisite information for processing.

In one embodiment, the third database 111 is a regulatory database that includes regulatory filing statuses of a PAC, regulatory dispatch dates, regulatory submission dates, and regulatory approval dates. The ATOMS module 104 extracts requisite information from database 109 in an automated format and relays the requisite information for processing.

The ATOMS module 104 utilizes one or more data handlers for acquiring data from the databases 107, 109, and 111. The data handlers retrieve the data from the separate databases and format the data in a standardized manner to enable the tracking and optimization of PACs, as described herein. The standardized format is a purpose-built data structure designed to facilitate tracking and optimization of PACs. The tracking and optimization enabled by the data structure includes sorting regulatory filing statuses according to their impact on product distribution, generating a lot genealogy to determine what product lots were consumed by downstream products, and sorting PAC-related data according to impact on product distribution.

The ATOMS module 104 uses a uniform data structure to enable tracking and optimization from a variety of sources. The uniform data structure for inventory data includes referencing each lot according to its internal lot number, as well as collecting its date of manufacture, site of manufacture, material type, and quantity of product yielded. For each inventory lot that consumes other materials, the lot number of each input material is also collected to form a drug genealogy. The uniform data structure for PAC data includes referencing each PAC by its identification number in its origin database, as well as collecting the material type, manufacturing site, and date of PAC implementation. The uniform data structure for PAC regulatory filing data includes referencing each filing by its identification number in its origin database, as well as collecting the PAC identification number that the filing is taking place in support of, the country in which the filing will take place, the type of filing that is to occur, the status of the regulatory filing, and the date of last update to the filing. In some embodiments, for all data structures listed above, the module stores or views this information in a database as a record in a single inventory table, or single record in a database query, depending on the data handler used.

In further embodiments, the ATOMS module 104 may include additional data handlers for acquiring data from databases other than the databases 107, 109, and 111. In one embodiment, a data handler is customized to acquire requisite data for tracking and optimization of PACs from different formats or sources. Requisite data includes, but is not limited to, PAC data, regulatory data, and inventory data. In some embodiments, requisite data is copied from data sources by the data handler, and stored in a uniform aggregation database within the ATOMS module, where it is used to enable the tracking and optimization of PACs. The aggregation database within the ATOMS module stores data from the various sources in a standardized format as to enable the analysis capabilities of the ATOMS module. In some embodiments, requisite data is copied into memory from data sources by the data handler, and stored in a uniform data structure within memory as the ATOMS module executes, where it is used to enable the tracking and optimization of PACs. At the termination of the ATOMS module execution, the data is released from memory. In some embodiments, requisite data is persistently accessed at the data source by the data handler using a database view and viewed in a uniform aggregation database within the ATOMS module, where it is used to enable the tracking and optimization of PACs. Because all data retrieved by a data handler is combined in a standardized format, the analysis capabilities of the ATOMS module can therefore be utilized for the tracking and optimization of PACs using any data source.

Once retrieved and formatted, the information from the databases 107, 109, and 111 may be compared by the ATOMS module 104, and a list of applicable PACs generated.

In some embodiments, the ATOMS module 104 uses SQL (structured query language) queries to obtain the data from the databases 107, 109, and 111. The ATOMS module 104 may also use SQL to query other data sources directly for requisite information.

In one embodiment, the ATOMS module 104 may utilize specific methods to obtain information from the database. In some embodiments, the ATOMS module 104 ascertains the impact of PACs on a drug genealogy. This may be accomplished through a process by which the date of manufacture (DOM) of each component in the genealogy is compared against the date of implementation (DOI) of a PAC impacting the CMO/process where the component was made to determine if the PAC impacts that particular lot of component.

In some embodiments, the ATOMS module 104 includes an application extension to run reports directly from an inventory management system that includes the first database 107, a PAC information system that includes the second database 109, and/or a regulatory management system that includes the third database 111. The ATOMS module 104 may further integrate additional SQL queries to other inventory databases to obtain drug genealogy and component manufacture dates.

In an embodiment, the ATOMS module 104 includes an SAP ERP extension to run reports directly from SAP. The ATOMS module may further integrate additional SQL queries to other inventory databases to obtain drug genealogy and component manufacture dates.

In an exemplary embodiment, the user-computing device 112 is a desktop, laptop, smartphone, tablet, or other computing device, used by an employee or a customer. The user-computing device 112 may include an application installed on the user-computing device 112 and/or a webpage displayed within a web browser that communicates with computing device 102 and ATOMS module 104 via a communications network 114. The user computing device 112 includes a dashboard 116 displayed within the application and/or the webpage.

The communications network 114 can be any network over which information can be transmitted between devices communicatively coupled to the network. For example, one or more portions of communications network 114 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

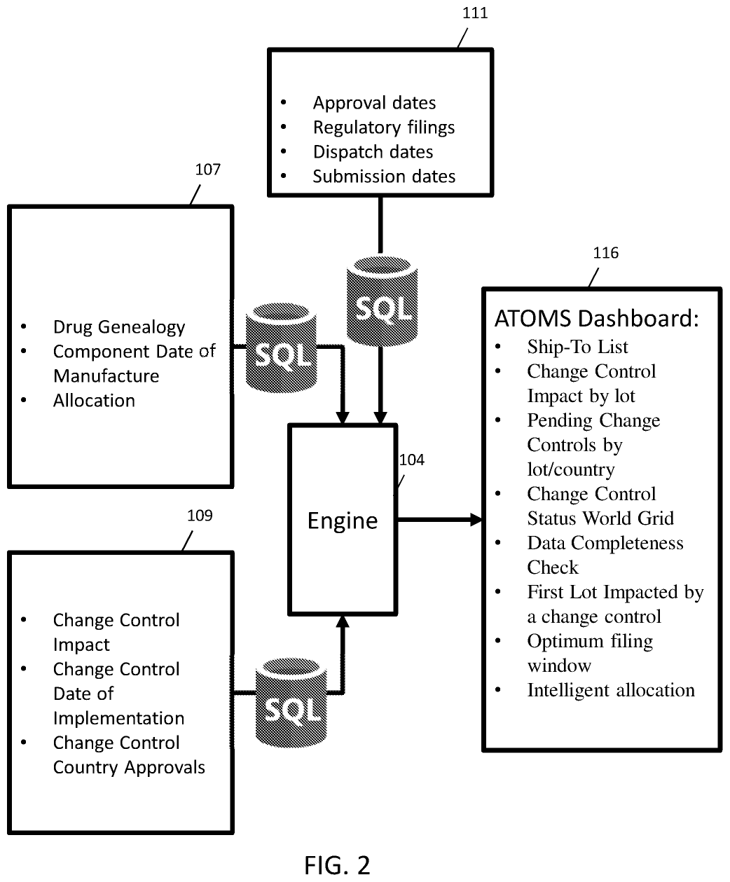

FIG. 2 illustrates an exemplary method 200 for processing data obtained from the first database 107, the second database 109, and/or the third database 111, and displaying results on a dashboard 116 using the ATOMS module 104. The first database 107 includes drug genealogy and component date of manufacture. The first database 107 may be, for example, an SAP Enterprise Resource Planner. The second database 109 includes PAC impact, PAC date of implementation, and PAC country approvals. The second database 109 may include the Trackwise™ platform. The second database 109 may also serve as a linked database platform including CATSWeb Quality Management System and Liquent Insight Regulatory Management System™. The third database 111 includes regulatory data, such as regulatory filing statuses of a PAC, regulatory dispatch dates, regulatory submission dates, and regulatory approval dates.

Continuing with the discussion of FIG. 2, the ATOMS module 104 obtains the information from the first database 107, the second database 109, and/or the third database 111. The ATOMS module 104 analyzes the information as described herein. The ATOMS module 104 transmits the results of the analysis to the ATOMS dashboard 116 displayed on the user-computing device 112. The ATOMS dashboard 116 may include, for example, a ship-to list, PAC impact by lot, pending PACs by lot/country, PAC status, first lot impacted by a PAC, optimum filing window for new PACs, and intelligent allocation.

In some embodiments, the ATOMS module 104 transmits data integrity alerts to the ATOMS dashboard 116. The data integrity alerts may include, for example, PAC missing date of implementation, PAC missing material indication, inconsistent filing/approval statuses, filing required/has approval date, invalid filing status, and missing filing status.

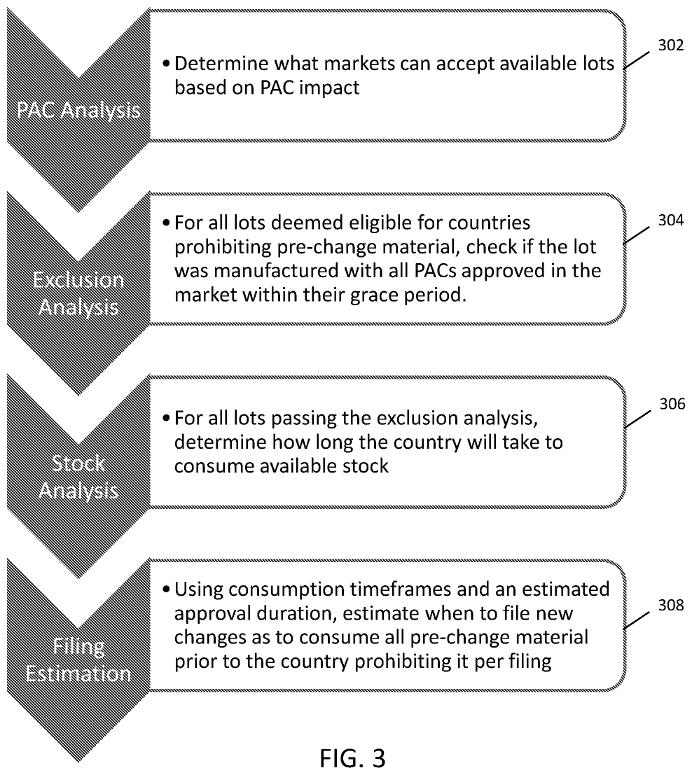

FIG. 3 illustrates a method to determine filing estimations, according to an exemplary embodiment. The method includes a PAC analysis 302, an exclusion analysis 304, a stock analysis 306, and a filing estimation 308. In the PAC analysis 302, the ATOMS module 104 determines what markets or countries can accept available lots based on PAC impact. In the exclusion analysis 304, for all lots deemed eligible for countries prohibiting pre-change material, the ATOMS module 104 determines whether the lot was manufactured with all PACs approved in the market, or within the grace period for any PACs currently required in the market but not implemented during manufacture. In the stock analysis 306, for all lots passing the exclusion analysis 304, the ATOMS module 104 determines how long the country will take to consume available stock. In the filing estimation 308, the ATOMS module 104 uses consumption timeframes and estimated approval duration to estimate when to file new changes as to consume all pre-change material prior to the country prohibiting it per filing. In a further embodiment the ATOMS module 104 executes a command for filing the new change in that country, e.g., to coordinate with consumption of the pre-change supply.

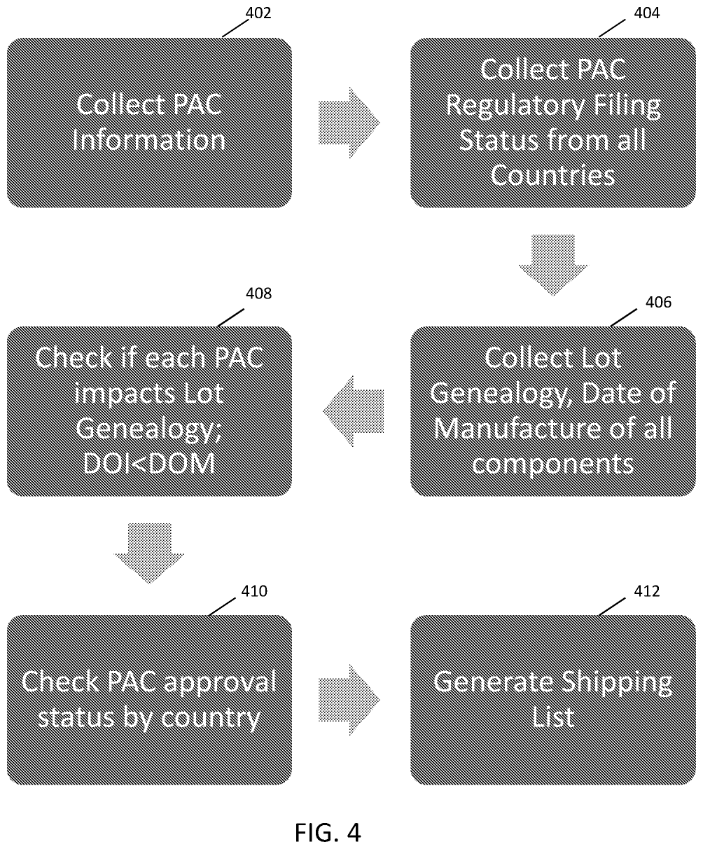

FIG. 4 illustrates a method to generate a shipping list, according to an exemplary embodiment. At 402, the ATOMS module 104 collects PAC information, including affected materials, equipment, and manufacturing sites from requisite data sources containing quality data. At 404, the ATOMS module 104 collects the regulatory filing status of each PAC for all countries from requisite data sources containing regulatory data.

At 406, the ATOMS module collects lot genealogy and date of manufacture of all components. At 408, the ATOMS module determines whether each PAC impacts lot genealogy (using the predefined ruleset described herein). At 410, the ATOMS module determines PAC approval status by country. At 412, the ATOMS module generates a shipping list of countries that can accept the analyzed product lot based on approval of applicable PACs.

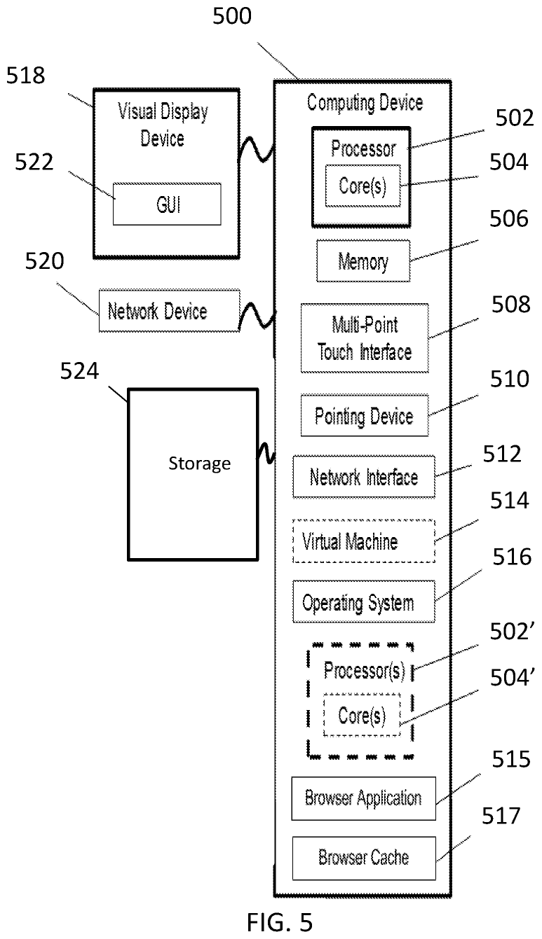

FIG. 5 is a block diagram of an example computing device 500 that can be used to perform one or more steps of the methods provided by exemplary embodiments. In an exemplary embodiment, computing device 500 is a computing device 102 as shown in FIG. 1 executing ATOMS module 104 and/or a user computing device 112 shown in FIG. 1. Computing device 500 includes one or more non-transitory computer-readable media for storing one or more computer-executable instructions or software for implementing exemplary embodiments described herein. The non-transitory computer-readable media can include, but are not limited to, one or more types of hardware memory, non-transitory tangible media (for example, one or more magnetic storage disks, one or more optical disks, one or more USB flashdrives), and the like. For example, a memory 506 included in computing device 500 can store computer-readable and computer-executable instructions or software for implementing exemplary embodiments described herein. Computing device 500 also includes a processor 502 and an associated core 504, and optionally, one or more additional processor(s) 502′ and associated core(s) 504′ (for example, in the case of computer systems having multiple processors/cores), for executing computer-readable and computer-executable instructions or software stored in memory 506 and other programs for controlling system hardware. Processor 502 and processor(s) 502′ can each be a single core processor or multiple core (504 and 504′) processor. Computing device 500 may also include a browser application 515 and a browser cache 517 to enable a user to information on computing device 500.

Virtualization can be employed in computing device 500 so that infrastructure and resources in the computing device can be shared dynamically. A virtual machine 514 can be provided to handle a process running on multiple processors so that the process appears to be using only one computing resource rather than multiple computing resources. Multiple virtual machines can also be used with one processor.

Memory 506 can include a computer system memory or random access memory, such as DRAM, SRAM, EDO RAM, and the like. Memory 506 can include other types of memory as well, or combinations thereof. In some embodiments, a customer can interact with computing device 500 through a graphical user interface (GUI) 522 associated with a visual display device 518, such as a touch screen display or computer monitor. Visual display device 518 may also display other aspects, elements and/or information or data associated with exemplary embodiments. Computing device 500 may include other I/O devices for receiving input from a customer, for example, a keyboard or any suitable multi-point touch interface 508, a pointing device 510 (e.g., a pen, stylus, mouse, or trackpad). The keyboard 508 and pointing device 510 may be coupled to visual display device 518. Computing device 500 may include other suitable conventional I/O peripherals.

Computing device 500 can also include one or more storage devices 524, such as a hard-drive, CD-ROM, or other computer readable media, for storing data and computer-readable instructions and/or software, which implements embodiments of the system, as described herein, or portions thereof. Exemplary storage device 524 can also store one or more storage devices for storing any suitable information required to implement exemplary embodiments.

Computing device 500 can include a network interface 512 configured to interface via one or more network devices with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 512 can include a built-in network adapter, network interface card, PCMCIA network card, card bus network adapter, wireless network adapter, USB network adapter, modem or any other device suitable for interfacing computing device 500 to any type of network capable of communication and performing the operations described herein. Moreover, computing device 500 can be any computer system, such as a workstation, desktop computer, server, laptop, handheld computer, tablet computer, mobile computing or communication device, or other form of computing or telecommunications device that is capable of communication and that has sufficient processor power and memory capacity to perform the operations described herein.

Computing device 500 can run any operating system 516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 516 can be run in native mode or emulated mode. In an exemplary embodiment, the operating system 516 can be run on one or more cloud machine instances.

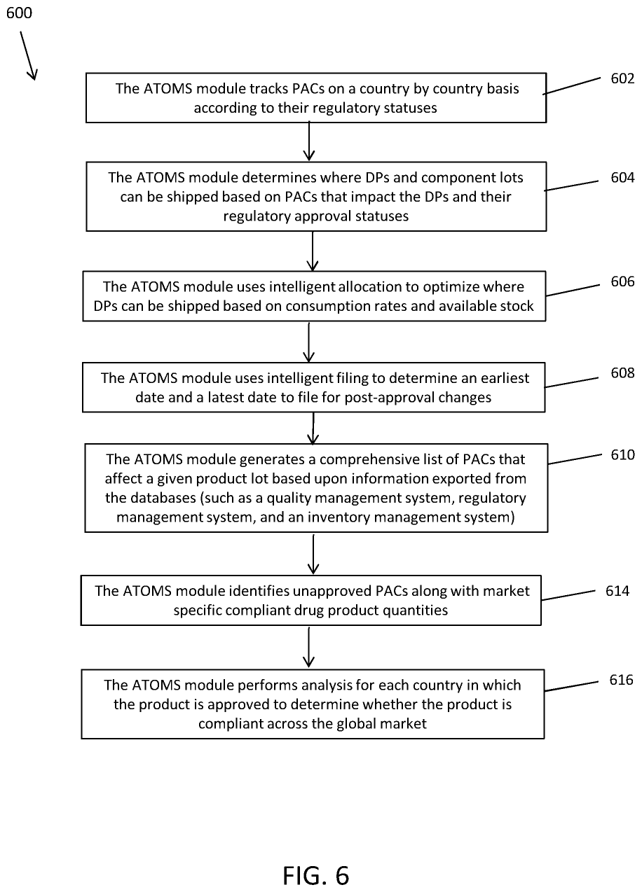

FIG. 6 is a method 600 for automated tracking and optimization of global manufacturing and supply based on PAC impact, in accordance with an exemplary embodiment. In step 602, the ATOMS module 104 tracks PACs on a country by country basis according to their regulatory statuses. In step 604, the ATOMS module 104 determines where DPs and component lots can be shipped based on PACs that impact the DPs and their regulatory approval statuses. In step 606, the ATOMS module 104 uses intelligent allocation to optimize where DPs can be shipped based on consumption rates and available stock. In step 608, the ATOMS module 104 uses intelligent filing to determine an earliest date and a latest date to file for post-approval changes. In step 610, the ATOMS module 104 generates a comprehensive list of PACs that affect a given product lot based upon information exported from databases (such as a quality management system, regulatory management system, and an inventory management system). In step 614, the ATOMS module 104 identifies unapproved PACs along with market specific compliant drug product quantities. In step 616, the ATOMS module 104 performs analysis for each country in which the product is approved to determine whether the product is compliant across the global market.

FIG. 7 illustrates a sample graphical user interface 700 displaying a list of lots that are invalid on a future date as a result of post-approval changes implemented during manufacturing, in accordance with an exemplary embodiment. For example, a country with no provisional grace period may have approved PAC numbers 33344 and 34357 on April 25^{th} thus invalidating lots 103552, 22300, 224221, 224222 and 224223 because of the market's requirement for the manufacture of product with all PACs currently approved by the regulatory authority of that market.

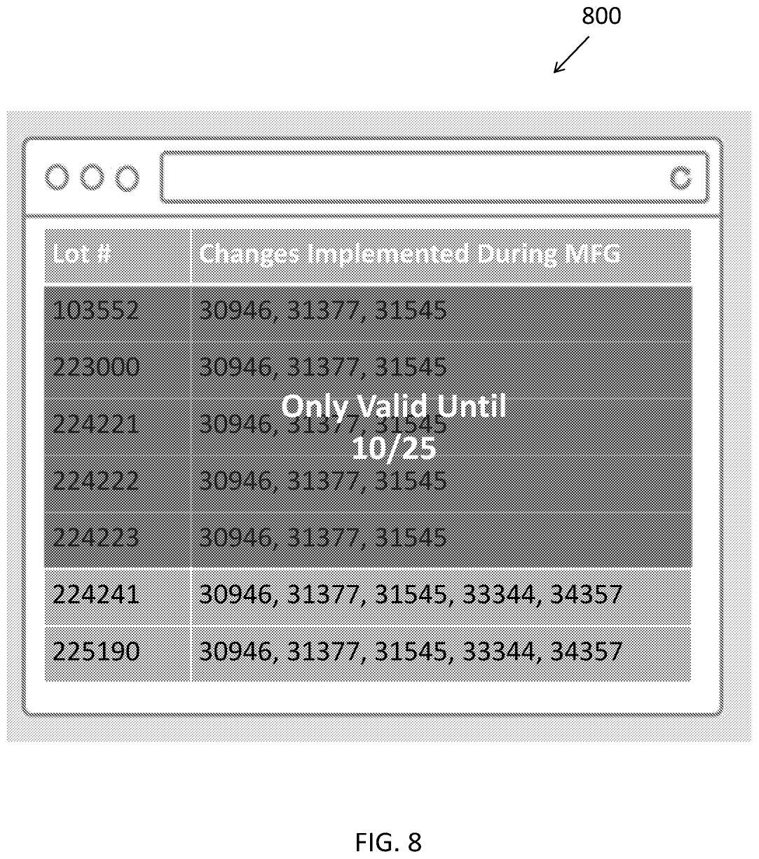

FIG. 8 illustrates a sample graphical user interface 800 displaying lots that are invalid on a future date as a result of changes implemented during manufacturing, in accordance with an exemplary embodiment. For example, a country with a 6-month provisional grace period may have approved PAC numbers 33344 and 34357 on April 25^{th}. Therefore, lots 103552, 22300, 224221, 224222 and 224223 can only be shipped to this sample market until October 25^{th} because of the market's requirement for the manufacture of product with all PACs currently approved by the regulatory authority of that market.

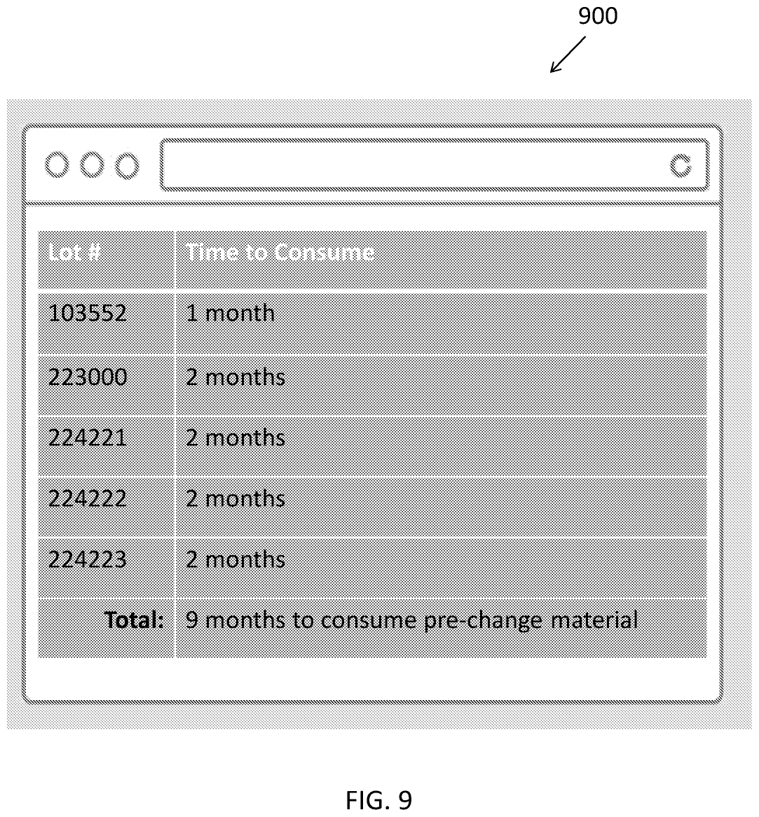

FIG. 9 illustrates a sample graphical user interface 900 displaying a length of time until lots are consumed and an optimal filing date for new changes. In the example, it will take a specified country 9 months to consume available stock of pre-change material according to current demand forecast with respect to forecast risk factor of consumption. To determine when to file for new changes in Japan, if the date is Apr. 24, 2018 and nine months is Jan. 24, 2019 when new material should be sent, and it takes Japan 3 months to approve changes according to current approval forecast with respect to forecast risk factor of approval, the target filing date for new changes is Oct. 24, 2018.

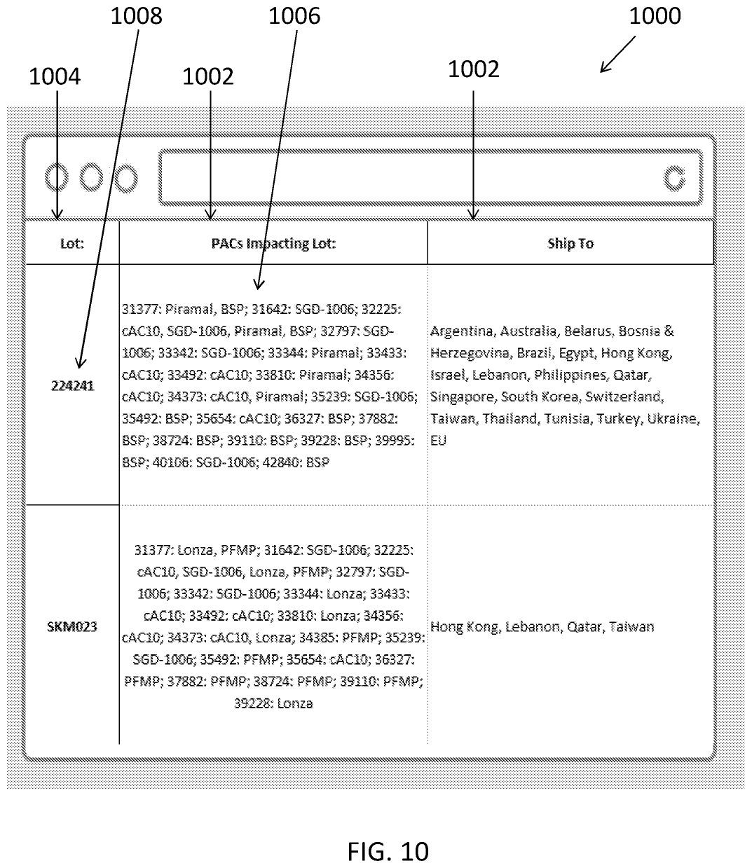

FIG. 10 illustrates a sample graphical user interface 1000 displaying lots impacted by post-approval changes. The graphical user interface 1000 displays a list of post-approval changes (shown in 1002) that impact specified lot numbers (shown in 1004). For example, the interface 1000 includes a list of post-approval changes (shown at 1006) that are associated with a specified lot number (shown at 1008). Based on an impact of the post-approval changes, the ATOMS module 104 determines one or more countries in which the lot associated with the lot number may be shipped (shown in 1006).

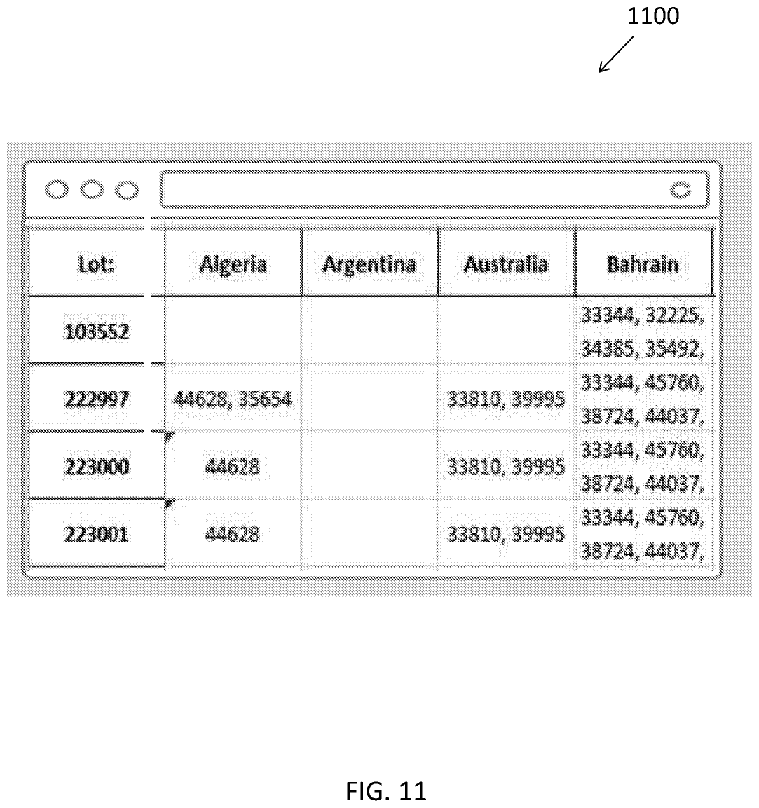

FIG. 11 illustrates a sample graphical user interface 1100 displaying pending PACs for specified lots in select countries, according to an exemplary embodiment. In further embodiments, the graphical user interface 1200 may display more or fewer countries and more or fewer lots.

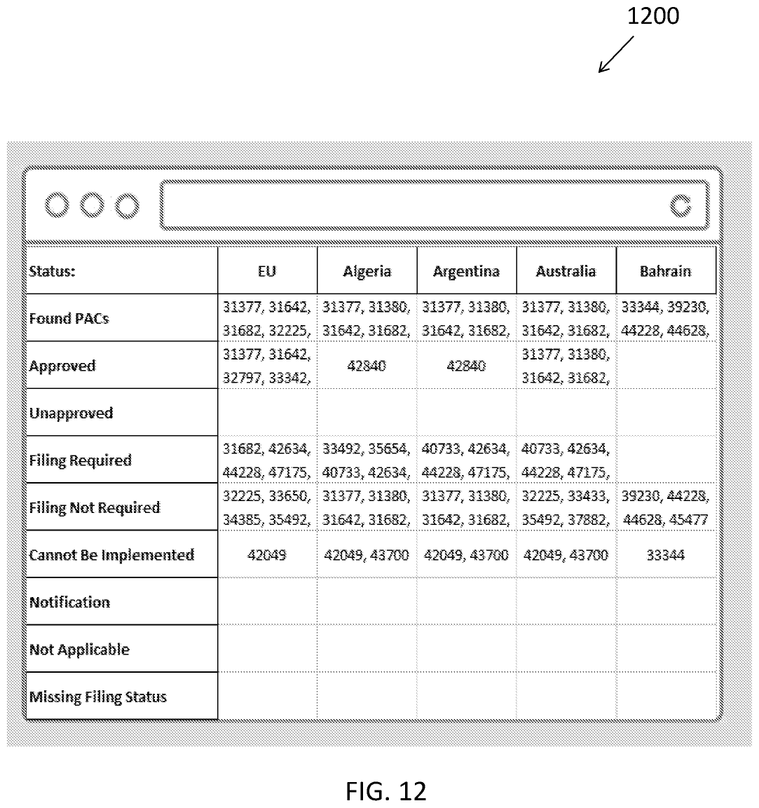

FIG. 12 illustrates a sample graphical user interface 1200 displaying PAC statuses in select countries, according to an exemplary embodiment. For example, the interface 1200 displays, for at least one country, PACs associated with the country, approved PACs associated with the country, unapproved PACs associated with the country, PACs needing to be filed in the country, PACs that have already been filed in the country or where filing is not needed in the country, and/or PACs that cannot be implemented in the country. In further embodiments, the graphical user interface 1200 may display more or fewer countries.

It should be appreciated that while a PAC is being assessed by a regulatory authority in a given market, product affected by the PAC cannot be sold in that market. Similarly, if a PAC has not yet been filed in a given market, product affected by the PAC cannot be sold in that market. If, at the end of the review process, the regulatory authority rejects the filing of a PAC, and does not approve the PAC, the regulatory authority may request further information to support the contents of the filing, or permanently reject the filing. If the PAC remains unapproved in the market, product affected by the PAC cannot be sold in that market. A regulatory authority may reject a filing for a number of reasons, including insufficient data to support the change described in the PAC, insufficient information provided about the nature of the change described in the PAC, or outstanding questions about the nature of the change described in the PAC.

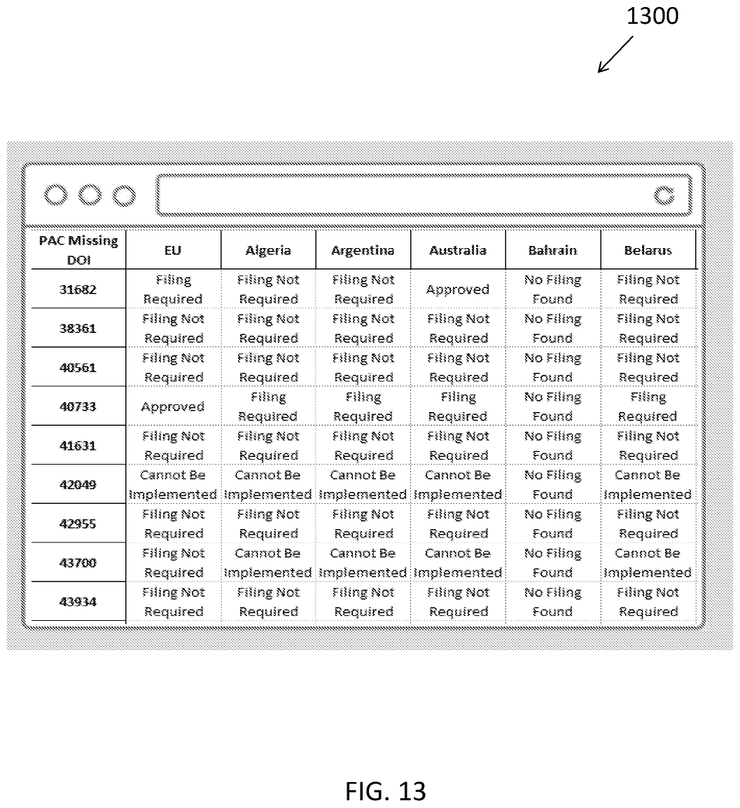

FIG. 13 illustrates a sample graphical user interface 1300 displaying filing statuses of PACs in select countries, according to an exemplary embodiment. The interface 1300 displays, for each select country, a filing status of one or more PACs. The filing status may include a specified PAC needs to be filed in that country, the specified PAC does not need to be in filed that country, the specified PAC was approved in that country, the specified PAC cannot be implemented in that country, or the specified PAC has not been filed in that country. In further embodiments, the graphical user interface 1200 may display more or less countries.

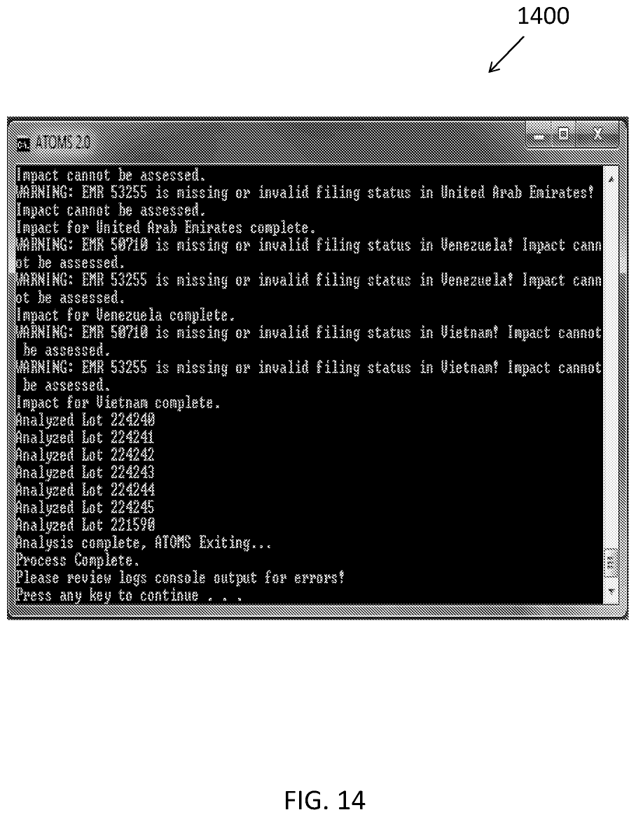

FIG. 14 illustrates a sample graphical user interface 1400 displaying an execution of the analysis process performed by the ATOMS computing device, according to an exemplary embodiment.

In some embodiments, the pharmaceutical drug product being manufactured is selected from the group consisting of brentuximab vedotin, vedolizumab, and ixazomib. In one embodiment, the pharmaceutical product is brentuximab vedotin. Brentuximab vedotin is also known by its trade name ADCETRIS® (Seattle Genetics) and is approved for the treatment of classical Hodgkin lymphoma and T-cell lymphoma. Brentuximab vedotin is an antibody-drug conjugate comprising a chimeric IgG1 anti-CD30 antibody conjugated via a peptide linker to monomethyl auristatin E. In one embodiment, the pharmaceutical product is vedolizumab. Vedolizumab is also known by its trade name ENTYVIO® (Millennium Pharmaceuticals, Inc.) and is approved for the treatment of moderately to severely active ulcerative colitis and Crohn's disease. Vedolizumab is a humanized antibody that binds □4□7 integrin. In one embodiment, the pharmaceutical product is ixazomib. Ixazomib is also known by its trade name NINLARO® (Millennium Pharmaceuticals, Inc.) and is approved for the treatment of multiple myeloma, e.g., in combination with lenalidomide and dexamethasone. Ixazomib, an oral inhibitor of the 20S proteasome, is a peptide boronic acid.

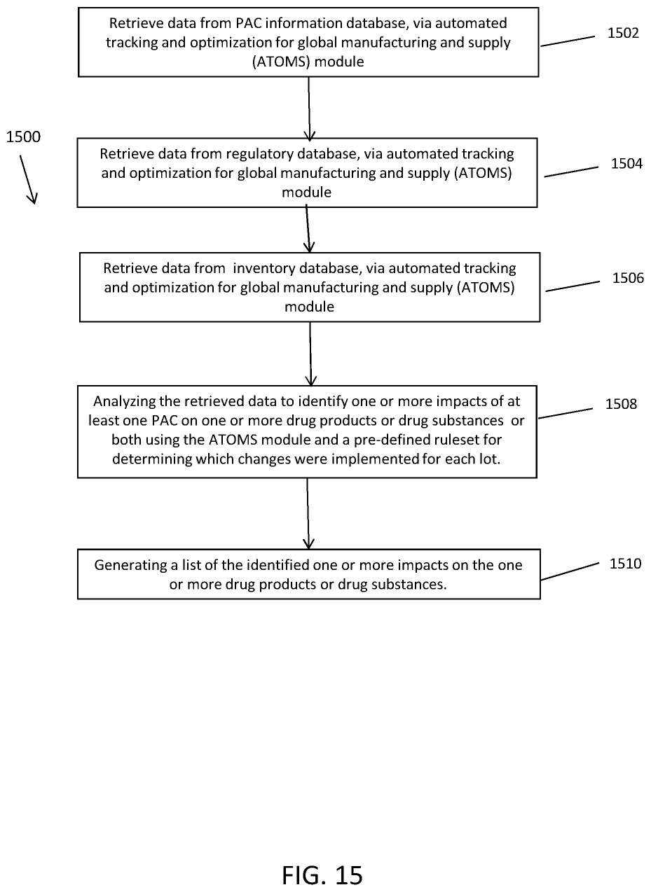

FIG. 15 is a method 1500 for automated tracking and optimization of global manufacturing and supply based on PAC impact, in accordance with an exemplary embodiment. In step 1502, the ATOMS module retrieves data from a PAC information database holding control information for a manufacturing process or material supply affected by one or more PACs In step 1504, the ATOMS module retrieves data from a regulatory database holding information relating to regulatory approvals by regulatory authorities of countries affected by the one or more PACs. In step 1506, the ATOMS module retrieves data from an inventory database holding lot information associated with drug products, drug substances or both. In some embodiments, steps 1502, 1504 and 1506 may take place in parallel, simultaneously and/or in a different order. In step 1508, the retrieved data is analyzed by the ATOMS module using a pre-defined ruleset for determining which changes were implemented for each lot to identify one or more impacts of at least one PAC on one or more drug products or drug substances. In step 1510, the ATOMS module generates a list of the identified one or more impacts on the one or more drug products or drug substances.

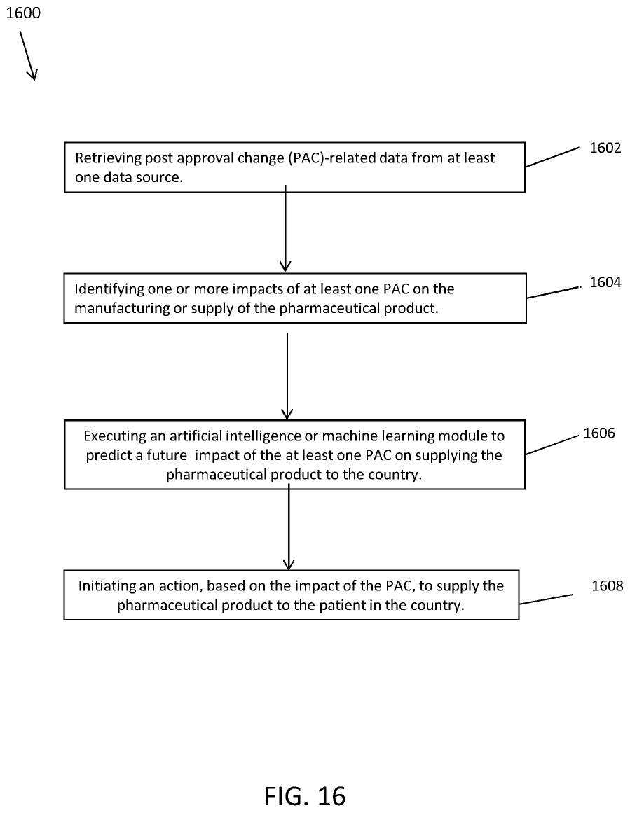

FIG. 16 is a method 1600 for automated tracking and optimization of global manufacturing and supply based on impacts of PACs, in accordance with an exemplary embodiment. In step 1602, the ATOMS module retrieves PAC-related data from at least one data source. In step 1604, the ATOMS module identifies one or more impacts of at least one PAC on the manufacturing or supply of the pharmaceutical product. In step 1606, the ATOMS module executes an artificial intelligence or machine learning module to predict a future impact of the at least one PAC on supplying the pharmaceutical product to the country. In step 1608, the ATOMS module initiates an action, based on the impact of the PAC, to supply the pharmaceutical product to the patient in the country.

The description herein is presented to enable any person skilled in the art to create and use a computer system configuration and related method and systems for automated tracking and optimization of PACs. Various modifications to the example embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention may be practiced without the use of these specific details. In other instances, well-known structures and processes are shown in block diagram form in order not to obscure the description of the invention with unnecessary detail. Thus, the present disclosure is not intended to be limited to the embodiments shown, but is to be accorded the widest scope consistent with the principles and features disclosed herein. For example, although embodiments have been described herein in terms of the ATOMS module interacting with three databases separately holding PAC data, inventory data and regulatory data, the information contained in the three databases may be combined or separated into a greater or lesser number of databases without departing from the scope of the present invention.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes multiple system elements, device components, or method steps, those elements, components, or steps can be replaced with a single element, component, or step. Likewise, a single element, component, or step can be replaced with multiple elements, components, or steps that serve the same purpose. Moreover, while exemplary embodiments have been shown and described with references to particular embodiments thereof, those of ordinary skill in the art will understand that various substitutions and alterations in form and detail can be made therein without departing from the scope of the invention. Further still, other aspects, functions, and advantages are also within the scope of the invention.

Portions or all of the embodiments of the present invention may be provided as one or more computer-readable programs or code embodied on or in one or more non-transitory mediums. The mediums may be, but are not limited to a hard disk, a compact disc, a digital versatile disc, a flash memory, a PROM, a RAM, a ROM, or a magnetic tape. In general, the computer-readable programs or code may be implemented in many computing languages.

Exemplary flowcharts are provided herein for illustrative purposes and are non-limiting examples of methods. One of ordinary skill in the art will recognize that exemplary methods can include more or fewer steps than those illustrated in the exemplary flowcharts, and that the steps in the exemplary flowcharts can be performed in a different order than the order shown in the illustrative flowcharts.

We claim:

1. A computing device-implemented method for automated tracking and optimization of global manufacturing and supply based on impacts of post-approval changes (PACs), the computing device including one or more processors, the method comprising:
retrieving data from data sources, via an automated tracking and optimization for global manufacturing and supply (ATOMS) module, the data sources including at least an inventory database, a regulatory database, and a PAC information database, the inventory database holding lot information associated with drug products, drug substances or both, the PAC information database holding control information for a manufacturing process or material supply affected by one or more PACs, the regulatory database holding information relating to regulatory approvals by regulatory authorities of a plurality of countries of the one or more PACs;
analyzing the retrieved data to identify one or more impacts of at least one PAC on one or more drug products or drug substances using the ATOMS module and a pre-defined ruleset for determining which changes were implemented for each lot; and
generating a list of the identified one or more impacts on the one or more drug products or drug substances.

2. The method of claim 1 wherein the list is generated by set notation.

3. The method of claim 1, wherein the analyzing further includes:
analyzing consumption data relating to drug products or drug substances in each of the plurality of countries;
identifying, based on the analyzing, at least one country of the plurality of countries in which an existing stock of drug products or drug substances is approaching an exhausted status; and
executing an artificial intelligence or machine learning module to preemptively ascertain the impact of the at least one PAC on upstream component lots used to manufacture the drug products or drug substances.

4. The method of claim 3, wherein the analyzing uses intelligent allocation.

5. The method of claim 3 or 4, wherein based on the ascertained impact, at least one upstream component lot is identified for accelerated shipment downstream for manufacturing so as to address demand in the at least one country in which the stock of drug products or drug substances is approaching an exhausted status.

6. The method of claim 3 or 4, wherein based on the ascertained impact, raw materials are ordered for manufacturing lots.

7. The method of claim 3 or 4, wherein based on the ascertained impact, an instruction to alter a shipping schedule is generated based on the approaching exhausted status of stock in the at least one country.

8. The method of claim 3 or 4, wherein based on the ascertained impact, a timing of a regulatory filing related to a PAC is scheduled.

9. The method of claim 8 wherein the ascertaining the impact uses intelligent filing.

10. The method of claim 3 or 4, wherein based on the ascertained impact, an instruction to commence manufacturing a drug substance or drug product is generated.

11. The method of claim 1, further comprising:
displaying the list of the identified one or more impacts on the one or more drug products or drug substances via a graphical user interface provided as a visual dashboard on a display surface.

12. The method of claim 1, wherein the list of the identified one or more impacts on the one or more drug products or drug substances identifies countries associated with each of the one or more PACs.

13. The method of claim 12, wherein the list of the identified one or more impacts on the one or more drug products or drug substances indicates a PAC approval status or filing status for each of the identified countries.

14. The method of claim 13, wherein the list of the identified one or more impacts identifies locations where drug product, drug substance, active pharmaceutical ingredients, or other component lots can be shipped based on the PAC approval statuses of each of the identified countries.

15. The method of any one of claims 1 to 14, wherein the list is translated into a barcode affixed to the drug product, drug substance, active pharmaceutical ingredients, or other component lots.

16. The method of claim 1, further comprising:
determining a length of time until a drug product lot is consumed in one or more specified countries of the plurality of countries; and
determining an earliest date and a latest date to file for a PAC in the one or more specified countries based on an expected length of time until at least one product lot is consumed.

17. The method of any one of claims 1 to 16, further comprising:
transmitting instructions to control a manufacturing device or an inventory management device to perform a manufacturing process or a labeling process based on the analysis.

18. The method of any ones of claims 1 to 17, further comprising:
identifying a date on which a drug substance or drug product will become invalid as a result of at least one of the one or more PACs.

19. A computing device-implemented method of supplying a pharmaceutical product to a patient in a country regulated by a pharmaceutical regulatory agency, the computing device including one or more processors, the method comprising:
retrieving post approval change (PAC)-related data from at least one data source;
identifying one or more impacts of at least one PAC on the manufacturing or supply of the pharmaceutical product;
executing an artificial intelligence or machine learning module to predict a future impact of the at least one PAC on supplying the pharmaceutical product to the country; and
initiating an action, based on the impact of the PAC, to supply the pharmaceutical product to the patient in the country.

20. The method of claim 19, wherein the action comprises at least one activity selected from the group consisting of a regulatory filing, shipping, procurement of raw materials, manufacturing DS or DP and country-specific packaging or labeling.

21. The method of claim 19, wherein the impact of the PAC is a regulatory status or regulatory requirement of the PAC.

22. The method of claim 19, wherein the pharmaceutical product is selected from the group consisting of brentuximab vedotin, vedolizumab and ixazomib.

23. The method of claim 19, wherein the at least one PAC-related data source is selected from the group consisting of an inventory database, a regulatory database, and a PAC information database.

24. The method of claim 23, wherein the inventory database holds lot information associated with drug products, drug substances or both, the PAC information database holds control information for a manufacturing process or material supply affected by one or more PACs, and the regulatory database holds information relating to regulatory approvals by regulatory authorities of one or a plurality of countries.

25. The method of claim 19, wherein identifying one or more impacts of at least one PAC on the pharmaceutical product comprises:
analyzing the retrieved data using a pre-defined ruleset for determining which changes were implemented for each lot of the pharmaceutical product.

26. The method of claim 19, wherein ascertaining the impact of the at least one PAC comprises:
identifying upstream component lots which should accelerate shipment downstream for manufacturing so as to address demand in the at least one country in which the stock of the pharmaceutical product is approaching an exhausted status.

27. The method of any one of claims 19-26, further comprising:
displaying a list of the identified one or more impacts on the one or more drug products or drug substances via a graphical user interface provided as a visual dashboard on a display surface.

28. The method of claim 27, wherein the list of the identified one or more impacts on the one or more drug products or drug substances identifies countries associated with each of the one or more PACs.

29. The method of claim 28, wherein the list of the identified one or more impacts on the one or more drug products or drug substances indicates a PAC approval status or filing status for each of the identified countries.

30. The method of claim 28, wherein the list of the identified one or more impacts identifies locations where drug product, drug substance, active pharmaceutical ingredients, or other component lots can be shipped based on the PAC approval statuses of each of the identified countries.

31. The method of any one of claims 1 to 18 and 27 to 30, wherein the list is translated into a barcode affixed to the drug product, drug substance, active pharmaceutical ingredients, or other component lots.

32. The method of any one of claims 1 to 31, further comprising:
determining a length of time until a drug product lot is consumed in one or more specified countries of the plurality of countries; and
determining an earliest date and a latest date to file for a PAC in the one or more specified countries based on an expected length of time until the at least one product lot is consumed.

33. The method of any one of claims 1 to 32, further comprising:
transmitting instructions to control a manufacturing device or an inventory management device to perform a manufacturing process or a labeling process based on the analysis.

34. The method of any ones of claims 1 to 33, further comprising:
identifying a date on which a drug substance or drug product will become invalid as a result of at least one of the one or more PACs.

35. A system for automated tracking and optimization of global manufacturing and supply based on impacts of post-approval changes (PACs), the system comprising:
an inventory database holding lot information associated with drug products, drug substances or both;
a regulatory database holding information relating to regulatory approvals by regulatory authorities of a plurality of countries of the one or more PACs;
a PAC information database holding control information for a manufacturing process or material supply for at least one of a drug substance and drug product affected by one or more PACs; and
a computing device equipped with one or more processors and configured to execute an automated tracking and optimization for global manufacturing and supply (ATOMS) module, the ATOMS module when executed:
retrieving data from data sources, including the inventory database, regulatory database and PAC information database;
analyzing the retrieved data to identify one or more impacts of at least one PAC on one or more drug products or drug substances and a pre-defined ruleset for determining which changes were implemented for each lot; and
generating a list of the identified one or more impacts on the one or more drug products or drug substances.

36. The system of claim 35, wherein the ATOMS module when executed further:
analyzes consumption data relating to drug products or drug substances in each of the plurality of countries;
identifies, based on the analyzing, at least one country of the plurality of countries in which an existing stock of drug products or drug substances is approaching an exhausted status; and
executes an artificial intelligence or machine learning module to preemptively ascertain the impact of the at least one PAC on upstream component lots used to manufacture the drug products or drug substances.

37. The system of claim 36, wherein based on the ascertained impact, the ATOMS module when executed identifies at least one upstream component lot for accelerated shipment downstream for manufacturing so as to address demand in the at least one country in which the stock of drug products or drug substances is approaching an exhausted status.

38. The system of claim 36, wherein based on the ascertained impact, the ATOMS module when executed transmits orders for raw materials for manufacturing lots.

39. The system of claim 36, wherein based on the ascertained impact, the ATOMS module when executed generates an instruction to alter a shipping schedule based on the approaching exhausted status of stock in the at least one country.

40. The system of claim 36, wherein based on the ascertained impact, the ATOMS module when executed schedules a timing of a regulatory filing related to a PAC.

41. The system of claim 36, wherein based on the ascertained impact, the ATOMS module when executed generates an instruction to commence manufacturing a drug substance or drug product.

42. The system of claim 35, wherein the ATOMS module when executed further:
generates a display of the list of the identified one or more impacts on the one or more drug products or drug substances via a graphical user interface provided as a visual dashboard on a display surface.

43. The system of claim 35, wherein the list of the identified one or more impacts on the one or more drug products or drug substances identifies countries associated with each of the one or more PACs.

44. The system of claim 43, wherein the list of the identified one or more impacts on the one or more drug products or drug substances indicates a PAC approval status or filing status for each of the identified countries.

45. The system of claim 44, wherein the list of the identified one or more impacts identifies locations where drug product, drug substance, active pharmaceutical ingredients, or other component lots can be shipped based on the PAC approval statuses of each of the identified countries.

46. The system of any one of claims 32 to 45, wherein the list is translated into a barcode affixed to the drug product, drug substance, active pharmaceutical ingredients, or other component lots.

47. The system of claim 35, wherein the ATOMS module when executed further:
determines a length of time until a drug product lot is consumed in one or more specified countries of the plurality of countries; and
determines an earliest date and a latest date to file for a PAC in the one or more specified countries based on an expected length of time until the at least one product lot is consumed.

48. The system of claims 35 to 47, wherein the ATOMS module when executed further:
transmits instructions to control a manufacturing device or an inventory management device to perform a manufacturing process or a labeling process based on the analysis.

49. The system of claims 35 to 48, wherein the ATOMS module when executed further:
identifies a date on which a drug substance or drug product will become invalid as a result of at least one of the one or more PACs.

50. A non-transitory medium storing instructions for automated tracking and optimization of global manufacturing and supply based on impacts of post-approval changes (PACs), the instructions executable by at least one computing device equipped with one or more processors, the instructions when executed causing the at least one computing device to:
retrieve data from data sources, via an automated tracking and optimization for global manufacturing and supply (ATOMS) module, the data sources including at least an inventory database, a regulatory database, and a PAC information database, the inventory database holding lot information associated with drug products, drug substances or both, the PAC information database holding control information for a manufacturing process or material supply affected by one or more PACs, the regulatory database holding information relating to regulatory approvals by regulatory authorities of a plurality of countries of the one or more PACs;
analyze the retrieved data to identify one or more impacts of at least one PAC on one or more drug products or drug substances using the ATOMS module and a pre-defined ruleset for determining which changes were implemented for each lot; and
generate a list of the identified one or more impacts on the one or more drug products or drug substances.

51. The medium of claim 50, wherein the analyzing further includes:
analyzing consumption data relating to drug products or drug substances in each of the plurality of countries;
identifying, based on the analyzing, at least one country of the plurality of countries in which an existing stock of drug products or drug substances is approaching an exhausted status; and
executing an artificial intelligence or machine learning module to preemptively ascertain the impact of the at least one PAC on upstream component lots used to manufacture the drug products or drug substances.

52. The medium of claim 51, wherein based on the ascertained impact, at least one upstream component lot is identified for accelerated shipment downstream for manufacturing so as to address demand in the at least one country in which the stock of drug products or drug substances is approaching an exhausted status.

53. The medium of claim 51, wherein based on the ascertained impact, raw materials are ordered for manufacturing lots.

54. The medium of claim 51, wherein based on the ascertained impact, an instruction to alter a shipping schedule is generated based on the approaching exhausted status of stock in the at least one country.

55. The medium of claim 51, wherein based on the ascertained impact, a timing of a regulatory filing related to a PAC is scheduled.

56. The medium of claim 50, wherein based on the ascertained impact, an instruction to commence manufacturing a drug substance or drug product is generated.

57. The medium of claim 50, further comprising:
displaying the list of the identified one or more impacts on the one or more drug products or drug substances via a graphical user interface provided as a visual dashboard on a display surface.

58. The medium of claim 50, wherein the list of the identified one or more impacts on the one or more drug products or drug substances identifies countries associated with each of the one or more PACs.

59. The medium of claim 58, wherein the list of the identified one or more impacts on the one or more drug products or drug substances indicates a PAC approval status or filing status for each of the identified countries.

60. The medium of claim 59, wherein the list of the identified one or more impacts identifies locations where drug product, drug substance, active pharmaceutical ingredients, or other component lots can be shipped based on the PAC approval statuses of each of the identified countries.

61. The medium of claims 50 to 60, wherein the list is translated into a barcode affixed to the drug product, drug substance, active pharmaceutical ingredients, or other component lots.

62. The medium of claim 50, further comprising:
determining a length of time until a drug product lot is consumed in one or more specified countries of the plurality of countries; and
determining an earliest date and a latest date to file for a PAC in the one or more specified countries based on an expected length of time until the at least one product lot is consumed.

63. The medium of claims 50 to 62, further comprising:
transmitting instructions to control a manufacturing device or an inventory management device to perform a manufacturing process or a labeling process based on the analysis.

64. The medium of claims 50 to 63, further comprising:
identifying a date on which a drug substance or drug product will become invalid as a result of at least one of the one or more PACs.
